(19) United States
(12) Patent Application Publication
(10) Pub. No.: US 2021/0317181 A1
(43) Pub. Date: Oct. 14, 2021

(54) UNIVERSAL CHIMERIC RECEPTORS

(71) Applicant: Asimov, Inc., Cambridge, MA (US)

(72) Inventor: Alec Andrew Nielsen, Cambridge, MA (US)

(73) Assignee: Asimov, Inc., Cambridge, MA (US)

(21) Appl. No.: 17/264,470

(22) PCT Filed: Aug. 2, 2019
(86) PCT No.: PCT/US19/44784

§ 371 Date: Jan. 29, 2021

Related U.S. Application Data
(60) Provisional application No. 62/713,731, filed on Aug. 2, 2018.

(51) Int. Cl. C07K 14/725; C07K 14/705; A61K 35/17

(52) U.S. Cl. CPC C07K 14/7051; C07K 14/70521; A61K 35/17; C07K 2319/02; C07K 2319/10; C07K 2319/92; C07K 2319/03; C07K 14/70578

(57) ABSTRACT

Disclosed herein are chimeric receptors and precursors, chimeric antigen receptors and precursors, universal chimeric receptor cell precursors, chimeric antigen receptor T cells, and methods of constructing and using the same.

### RELATED APPLICATION

This application claims the benefit under 35 U.S.C. § 119 of U.S. provisional application Ser. No. 62/713,731, filed Aug. 2, 2018, the entire contents of which are incorporated by reference herein.

### FIELD

Disclosed herein are chimeric receptors and precursors, chimeric antigen receptors and precursors, universal chimeric receptor cell precursors, chimeric antigen receptor T cells, and methods of constructing and using the same.

### BACKGROUND

Chimeric receptor proteins are useful in cell-based therapies, however, they remain challenging and expensive to construct and express in relevant cells.

### SUMMARY

As disclosed herein, universal chimeric receptor proteins can be made by providing two parts of such chimeric receptors, and utilizing protein splicing to join the two parts to form a chimeric receptor protein.

According to one aspect, chimeric receptor precursors are provided that include an extracellular domain comprising a protein splicing domain, a transmembrane domain, and an intracellular effector domain.

In some embodiments, the protein splicing domain comprises one part of a split protein-splicing domain. In some embodiments, the split protein-splicing domain is a split intein. In some embodiments, the split intein is a portion of a maxi-intein, a mini-intein, a trans-splicing intein, or an alanine intein.

In some embodiments, the protein splicing domain comprises a protein or peptide that forms a spontaneous isopeptide bond. In some embodiments, the protein or peptide that forms a spontaneous isopeptide bond is SpyCatcher protein, a SpyTag peptide, a

SnoopCatcher protein, a SnoopTag peptide, a SdyCatcher protein, a SdyTag peptide or a first or second portion of a split CnaB domain.

In some embodiments, the protein splicing domain comprises a site recognized by a peptide ligase. In some embodiments, the peptide ligase is a sortase, a butelase, a subtiligase, a peptiligase or an omniligase.

In some embodiments, the chimeric receptor precursor is a chimeric antigen receptor precursor, an antigen receptor precursor, a cytokine receptor precursor, a growth factor receptor precursor, a chemokine receptor precursor, a hormone receptor precursor, an amino acid receptor precursor, a small molecule receptor precursor, an integrin precursor, a T-cell receptor precursor, a ligand-gated ion channel precursor, an Fc receptor precursor, or a G-protein coupled receptor precursor. In some embodiments, the chimeric receptor precursor is a chimeric antigen receptor precursor.

In some embodiments, the intracellular effector domain comprises a CD3zeta domain. In some embodiments, the intracellular effector domain further comprises one or more costimulatory domains. In some embodiments, the one or more costimulatory domains is selected from the group consisting of 4-1BB, CD28, CD27, ICOS, OX40, CD40, and GITR.

In some embodiments, the transmembrane domain is a CD28 transmembrane domain.

In some embodiments, the protein splicing domain is at the terminus of the extracellular domain distal to the transmembrane domain. In some embodiments, the protein splicing domain is at the amino terminus of the extracellular domain.

In some embodiments, the extracellular domain does not comprise a ligand binding domain.

In some embodiments, the intracellular effector domain is an intracellular signaling domain. In some embodiments, the intracellular signaling domain comprises a tyrosine kinase domain, a serine/threonine kinase domain, a histidine kinase domain, a CD3zeta domain, or a CD28 domain.

In some embodiments, the intracellular effector domain is a protein-binding domain.

According to another aspect, chimeric receptor targeting domains are provided that include a ligand binding domain, and a protein splicing domain.

In some embodiments, the protein splicing domain comprises one part of a split protein-splicing domain. In some embodiments, the split protein-splicing domain is a split intein. In some embodiments, the split intein is a portion of a maxi-intein, a mini-intein, a trans-splicing intein, or an alanine intein.

In some embodiments, the protein splicing domain comprises a protein or peptide that forms a spontaneous isopeptide bond. In some embodiments, the protein or peptide that forms a spontaneous isopeptide bond is SpyCatcher protein, a SpyTag peptide, a SnoopCatcher protein, a SnoopTag peptide, a SdyCatcher protein, a SdyTag peptide or a first or second portion of a split CnaB domain.

In some embodiments, the protein splicing domain comprises a site recognized by a peptide ligase. In some embodiments, the peptide ligase is a sortase, a butelase, a subtiligase, a peptiligase or an omniligase.

In some embodiments, the ligand binding domain is a chimeric antigen binding domain, an antigen binding domain, a cytokine binding domain, a growth factor binding domain, a chemokine binding domain, a hormone binding domain, amino acid binding domain, small molecule binding domain, an integrin binding domain, a T-cell receptor binding domain, a ligand-gated ion channel extracellular domain, an Fc receptor binding domain, or a G-protein coupled receptor binding domain. In some embodiments, the antigen binding domain comprises or consists of an scFv, a single domain antibody, a Fab, a SynNotch receptor, or a chimeric protein domain.

In some embodiments, the protein splicing domain is at a terminus of the chimeric receptor targeting domain. In some embodiments, the protein splicing domain is at the carboxy terminus of the chimeric receptor targeting domain.

In some embodiments, the protein splicing domain is not at a terminus of the chimeric receptor targeting domain.

According to another aspect, chimeric receptors are provided that include an extracellular domain comprising a ligand binding domain and a scar from a protein splicing reaction, a transmembrane domain, and an intracellular signaling domain.

In some embodiments, the protein splicing reaction is splicing of two split protein-splicing domains. In some embodiments, the split protein-splicing domains are complementary portions of a split intein. In some embodiments, the split inteins are complementary portions of a maxi-intein, a mini-intein, a trans-splicing intein, or an alanine intein

In some embodiments, the protein splicing reaction is spontaneous isopeptide bond formation. In some embodiments, the spontaneous isopeptide bond formation is between a SpyCatcher protein and a SpyTag peptide, a SnoopCatcher protein and a SnoopTag peptide, a SdyCatcher protein and a SdyTag peptide, or a first and second portion of a split CnaB domain.

In some embodiments, the protein splicing reaction is splicing catalyzed by a peptide ligase. In some embodiments, the peptide ligase is a sortase, a butelase, a subtiligase, a peptiligase or an omniligase.

In some embodiments, the intracellular signaling domain comprises a CD3zeta domain.

In some embodiments, the intracellular signaling domain further comprises one or more costimulatory domains. In some embodiments, the one or more costimulatory domains is selected from the group consisting of 4-1BB, CD28, CD27, ICOS, OX40, CD40, and GITR.

In some embodiments, the transmembrane domain is a CD28 transmembrane domain.

In some embodiments, the antigen binding domain comprises or consists of an scFv, a single domain antibody, a Fab, a SynNotch receptor, or a chimeric protein domain.

In some embodiments, the scar comprises 1, 2, 3, 4, or 5 amino acids.

In some embodiments, the chimeric receptor is a chimeric antigen receptor and wherein the ligand binding domain is an antigen binding domain.

According to another aspect, universal chimeric receptor cell precursors are provided that include a cell engineered to express a chimeric receptor precursor as disclosed herein. In some embodiments, the cell is a human cell. In some embodiments, the human cell is produced in vitro from a human stem cell. In some embodiments, the cell is a T cell.

In some embodiments, expression of the chimeric receptor precursor is constitutive. In some embodiments, expression of the chimeric receptor precursor is inducible.

According to another aspect, methods of making the universal chimeric receptor cell precursors disclosed herein are provided, the methods including introducing into a cell a construct that expresses the chimeric receptor precursor disclosed herein.

In some embodiments, expression of the chimeric receptor precursor is constitutive. In some embodiments, expression of the chimeric receptor precursor is inducible.

According to another aspect, cells are provided that include one or more of the chimeric receptors disclosed herein. In some embodiments, the cell is a human cell. In some embodiments, the human cell is produced in vitro from a human stem cell. In some embodiments, the cell is a T cell.

According to another aspect, methods of making the aforementioned cells are provided, the methods including introducing into a cell a construct that expresses any of the chimeric receptors disclosed herein.

According to another aspect, methods of making a cell containing a chimeric receptor are provided, the methods including contacting one or more of the universal chimeric receptor cell precursors disclosed herein with one or more of the chimeric receptor targeting domain disclosed herein for a time sufficient to splice or ligate together the one or more universal chimeric receptor precursor and the one or more chimeric receptor targeting domain to form a chimeric receptor cell, wherein the one or more receptor targeting domain includes a protein splicing domain that is complementary to the one or more protein splicing domain of the one or more universal chimeric receptor precursor, and wherein the one or more universal chimeric receptor precursor and the one or more chimeric receptor targeting domain are spliced or ligated together via their respective protein splicing domains.

In some embodiments, the protein splicing domains comprise complementary portions of a split protein-splicing domain. In some embodiments, the split protein-splicing domains are complementary portions of a split intein. In some embodiments, the split inteins are complementary portions of a maxi-intein, a mini-intein, a trans-splicing intein, or an alanine intein.

In some embodiments, the protein splicing domains comprise proteins or peptides that form a spontaneous isopeptide bond. In some embodiments, the proteins or peptides that form a spontaneous isopeptide bond are a SpyCatcher protein and a SpyTag peptide, a SnoopCatcher protein and a SnoopTag peptide, a SdyCatcher protein and a SdyTag peptide, or a first and second portion of a split CnaB domain.

In some embodiments, if the protein splicing domain comprises a site recognized by a peptide ligase and the protein splicing reaction is splicing catalyzed by a peptide ligase, then the methods further include contacting the universal chimeric antigen receptor T cell precursor and the chimeric antigen receptor targeting domain with a peptide ligase. In some embodiments, the peptide ligase is a sortase, a butelase, a subtiligase, a peptiligase or an omniligase.

In some embodiments, the cell is a human cell. In some embodiments, the human cell is produced in vitro from a human stem cell. In some embodiments, the cell is a T cell.

In some embodiments, the universal chimeric receptor cell precursor and the chimeric receptor targeting domain are contacted in vitro.

In some embodiments, the universal chimeric antigen cell precursor and the chimeric receptor targeting domain are contacted in vivo.

In some embodiments, the universal chimeric receptor cell precursor and the chimeric receptor targeting domain are contacted ex vivo.

In some embodiments, more than one receptor targeting domain is spliced to one or more types of universal chimeric receptor precursor, and wherein the protein splicing domains of the more than one receptor targeting domain are orthogonal.

According to another aspect, methods are provided, the methods including administering to a subject in need of such treatment a cell containing a chimeric receptor disclosed herein or a cell containing a chimeric receptor prepared by the methods disclosed herein. In some embodiments, the cell containing a chimeric receptor is a chimeric antigen receptor T (CAR-T) cell.

These and other aspects are descried in more detail below.

### DETAILED DESCRIPTION

As disclosed herein, universal chimeric receptor proteins can be made by providing two parts of such chimeric receptors, a “chimeric receptor precursor” and a “chimeric receptor targeting domain,” and utilizing protein splicing to join the two parts to form a complete chimeric receptor protein. The chimeric receptor precursor includes an extracellular domain comprising a protein splicing domain, a transmembrane domain and an intracellular effector domain, one or more (up to all) of which can be from the same or different receptor molecules (i.e., the extracellular domain, the transmembrane domain, and the intracellular effector domain can each be from a first, second, and/or third protein). The extracellular domain typically is itself chimeric in that the protein splicing domain is typically added to the remainder of the extracellular domain to facilitate protein splicing. The chimeric receptor targeting domain includes a ligand binding domain and a protein splicing domain that is compatible with the protein splicing domain of the extracellular domain, such as a split protein splicing domain that is a complementary portion of the split protein splicing domain of the extracellular domain. By “compatible with the protein splicing domain of the extracellular domain” is meant that the protein splicing domain of the extracellular domain and the protein splicing domain of the targeting domain are spliced together under suitable conditions. By a “complementary portion of a split protein-splicing domain” is meant that the two portions that are capable of splicing together when in proximity of each other. The targeting domain typically is itself chimeric in that the protein splicing domain is typically added to the remainder of the targeting domain (e.g., ligand binding domain) to facilitate protein splicing.

The chimeric receptor precursor and the chimeric receptor targeting domain are joined together by splicing of their respective protein splicing domains to form a chimeric receptor protein that includes an extracellular domain comprising a targeting domain (including a ligand binding domain), a transmembrane domain, and an intracellular effector domain. The protein splicing domains are completely or substantially removed during the splicing process; by “substantially removed” is meant that there may be a small number of amino acids (e.g., 1-5) left behind at the site of the protein splice, which small number of amino acids may be referred to as a “scar.”

As used herein, a “chimeric receptor” is a multidomain protein comprising an extracellular targeting domain, a transmembrane domain and an intracellular effector domain, in which the intracellular domain optionally has an intrinsic effector function, such as a kinase activity or other biochemical activity. In such chimeric receptors, binding of the extracellular targeting domain to its target ligand causes the intracellular effector domain to trigger one or more intracellular processes. Chimeric receptors can be engineered by combining wide variety of targeting domains, both rationally designed or naturally occurring with a wide variety of intracellular effector domains, either rationally designed or naturally occurring, to create novel proteins that detect extracellular ligands and trigger intracellular effects. The intracellular effects can occur via the effector function of the intracellular effector domain, or by binding to another, separate intracellular protein that carries out the effector function (e.g., a separate kinase).

In addition to chimeric receptors, the technology described herein can be used to produce engineered, spliced versions of non-chimeric receptors, i.e., in which the extracellular targeting domain, transmembrane domain and intracellular effector domain of the same receptor molecule are combined by protein splicing of a receptor precursor (having an extracellular domain that comprises a protein splicing domain, transmembrane domain, and intracellular effector domain) and a targeting domain (that comprises a ligand binding domain and a protein splicing domain), in which the two protein splicing domains facilitate splicing of the two proteins. Therefore, the descriptions of chimeric receptors will be understood to apply also to non-chimeric receptors.

The chimeric receptor assembly methods disclosed herein permit, for example, the use of a single chimeric receptor precursor with one targeting domain or more than one different targeting domains, each of which different targeting domains provides a different ligand-binding (e.g., targeting) function. Using such chimeric receptor precursors, cells can be prepared for later use with different targeting domains. For example, standardized cells expressing such chimeric receptor precursors can be prepared and used in many different situations, with particular binding specificity or specificities conferred by the selected targeting domain(s), which are added to the chimeric receptor precursors expressed by the standardized cells by protein splicing.

In embodiments where more than one different targeting domain is used to produce different chimeric receptors (e.g., using a cell containing a universal chimeric receptor precursor), the protein splicing domains of the different targeting domains can be orthogonal, and the chimeric receptor precursor can include more than one orthogonal splicing domain. For example, a first type of targeting domain having a first type of protein splicing domain and a second type of targeting domain having a second type of protein splicing domain can be spliced to a single type of chimeric receptor precursor that has two types of protein splicing domains that are complementary or compatible with the first and second types of protein splicing domains on the two types of targeting domains. Alternatively, the protein splicing domains of the different targeting domains can be orthogonal, and different types of chimeric receptor precursors that each have the complementary or compatible orthogonal splicing domain can be used. For example, a first type of targeting domain having a first type of protein splicing domain and a second type of targeting domain having a second type of protein splicing domain can be spliced to two types of chimeric receptor precursor that each have one type of protein splicing domain that is complementary or compatible with the first type or the second type of protein splicing domain on the two types of targeting domains.

### Protein Splicing Domains

In some aspects, the protein splicing domain of the extracellular domain is capable of spontaneously forming an isopeptide bond with another protein, such as a targeting domain, that includes a protein splicing domain that is compatible with the protein splicing domain of the extracellular domain. In some embodiments, the extracellular domain includes one part of a split protein splicing domain. The extracellular domain may include a portion of a split intein. As used herein, an “intein” is a domain of a protein that is capable of self-excision, resulting in joining of residual protein domains (“exteins”) via a peptide bond in a process known as protein splicing. Because of the functional analogy to gene transcript splicing, inteins are also sometimes described as “protein introns.” An intein can be a maxi-intein, or a mini-intein, or a trans-splicing intein, or an alanine splicing intein. A split intein is one of a pair of separately encoded and expressed polypeptides that typically comprise inteinic sequence and exteinic sequence. The pair of expressed polypeptides then interact to form a fusion peptide comprising the exteinic amino acid sequences of the constituent pair while excising the inteinic sequence. See, e.g., Wu, et al., Protein trans-splicing by a split intein encoded in a split DnaE gene of Synechocystis sp. PCC6803, Proc Natl Acad Sci USA, 1998 Aug. 4; 95 (16): 9226-9231. Those skilled in art of molecular or synthetic biology will thus appreciate that naturally occurring split intein pairs can be utilized to create genetically engineered self-assembling fusion proteins. In some embodiments, the splicing of proteins by such mechanism results in a “scar” (where a scar is residual sequence of the intein or other splicing domains, the residual sequence being in some cases 1-5 amino acids). In some embodiments, the splice between the chimeric receptor precursor and targeting domain is “scarless,” where “scarless” indicates that that the native amino acid sequences of two proteins are spliced without the inclusion of any amino acid residues that are exogenous to the two proteins. In other words, a scarless splice results in a fusion protein that does not have any residual amino acids from the protein splicing domains.

In some embodiments, the extracellular domain comprises one member of the following pairs of compatible protein splicing domains: a Spycatcher protein and a SpyTag peptide; a SnoopCatcher protein and a SnoopTag peptide; or a SdyCatcher protein and a SdyTag peptide, and the targeting domain has the other member of the pair of protein splicing domains.

SpyTag is a rationally-engineered peptide which spontaneously, rapidly, and with a high yield, forms a stable and effectively irreversible amide bond (remains intact on boiling with SDS) with a rationally engineered-protein partner SpyCatcher. The SpyTag and SpyCatcher pair were reverse engineered from naturally-occurring fibronectin binding protein (FbaB) in Streptococcus pyrogenes to provide an irreversible targetable locking mechanism as a tool to create novel protein architectures. See Zakeri, et al., Peptide tag forming a rapid covalent bond to a protein, through engineering a bacterial adhesion, Proc Natl Acad Sci USA, 2012 Mar. 20; 109 (12):E690-7; see also, e.g., the SpyTag/SpyCatcher

Protein Coupling Reagents commercially available from Kerafast, Boston, Mass., USA. Similarly, SnoopCatcher and SnoopTag are an engineered protein and peptide pair, based on adhesin RrgA from Streptococcus pneumoniae, that form a spontaneous covalent bond by an mechanism analogous to but orthogonal from the SpyCatcher/Tag tool. See Veggiani et al., Programmable polyproteams built using twin peptide superglues, PNAS Feb. 2, 2016. 113 (5) 1202-1207; available from Addgene, Cambridge, Mass., USA (e.g., Plasmid #72322). SdyTag and SdyCatcher, on the other hand, also utilize the self-assembling mechanism of fibronectin binding protein (FbaB), but in this case derived from Streptococcus dysgalactiae, creating an additional tool for directed covalent protein assembly that, although derived from the homolog protein in S. pyrogenes that underlies the SnoopCatcher/Tag system, manifests relatively low specificity for the cognate partner in the SpyCatcher/Tag system. See Tan, et al. (2016) Kinetic Controlled Tag-Catcher Interactions for Directed Covalent Protein Assembly, PLoS ONE 11 (10): e0165074.

In further embodiments, the extracellular domain comprises either of a first or second portion of the CnaB domain of the fibronectin-binding protein FbaB from Streptococcus spp other than S. pyrogenes or S. dysgalactiae, divided and optimized to take advantage of spontaneously-occurring isopeptide bonding, to create the elements of a two-part protein splicing mechanism. In such embodiments, the targeting domain comprises the other of the first or second portion of the CnaB domain than is present in the extracellular domain, such that the extracellular domain and targeting domain each comprises a compatible portion of the CnaB domain for protein splicing.

In some embodiments, the formation of the protein splice peptide bond requires the presence of an additional enzyme, cofactor, or molecular chaperone, for example a protein ligase. In such embodiments, the extracellular domain and the targeting domain are engineered to each include a sequence and/or structure recognized by the protein ligase. Non-limiting examples of protein ligases include a sortase, a butlease, a subtiligase, a peptiligase and an omniligase.

In some aspects, the protein splicing domain of the extracellular domain is engineered to comprise the end of the chimeric receptor precursor protein distal to the transmembrane domain, e.g., near or at the amino terminus. In some embodiments, the protein splicing domain is located at and includes the amino terminus of the chimeric receptor precursor. In some embodiments, the protein splicing domain of the targeting domain is engineered to comprise the C-terminal end of the targeting domain or to be C-terminal to the ligand binding domain such that the targeting domain is spliced to the extracellular domain protein distal to the transmembrane domain, i.e., in the order of: N-terminus-targeting domain-splice junction-transmembrane domain-intracellular effector domain-C-terminus.

In some embodiments, the protein splicing domains can both be located at the C-terminal end of the respective proteins, and these can be spliced using a sortase or an isopeptidase, which are capable of such “unnatural” fusions between two C-terminal tags (e.g., Wagner et al. Proc Natl Acad Sci U S A. 2014 Nov. 25; 111 (47): 16820-16825).

In some embodiments, isopeptide tags such as Spytag are inserted in the middle of a protein, for example between two subunits on a fusion protein.

### Transmembrane Domains

Transmembrane domains include amino acids suitable to cross cell membranes, joining an extracellular domain to an intracellular domain. In some embodiments, the transmembrane domain comprises a CD28 transmembrane domain, a CD3-zeta transmembrane domain, a CD4 transmembrane domain, a CD8 transmembrane domain or a 7-transmembrane domain (e.g., the transmembrane domain of a G-protein coupled receptor).

### Intracellular Effector Domains

Intracellular effector domains are joined to a transmembrane domain and reside in the interior of a cell. Intracellular effector domains have some effector function, which in some embodiments is an enzymatic function that transmits a signal to other molecules in the interior of the cell. The enzymatic function can be intrinsic to the effector domain, or extrinsic, in which case the effector domain typically has an amino acid sequence (e.g., an SH2 or SH3 sequence) that binds to an intracellular enzyme.

In some embodiments, the intracellular effector domain may be a CD3Zeta intracellular effector domain. The intracellular effector domain also in other embodiments comprises a CD3Zeta intracellular effector domain and a costimulatory domain. The costimulatory domain may include the 4-1BB costimulatory domain, the CD28 costimulatory domain, the CD27 costimulatory domain, the ICOS costimulatory domain, the OX40 costimulatory domain, the CD40 costimulatory domain, or the GITR costimulatory domain. In some embodiments, the intracellular portion of CD28 includes the terminal domain of the intracellular effector domain. In some embodiments, the intracellular effector domain comprises a signaling domain. The signaling domain may include tyrosine kinase, serine kinase, threonine kinase, and/or histidine kinase.

### Targeting Domains

Further disclosed herein are targeting domains that are engineered to be spliced onto the chimeric receptor precursors. The targeting domains include a ligand binding domain and a protein splicing domain. In some embodiments, the targeting domain protein splicing domain is a split-intein splicing domain, as described elsewhere herein. In some embodiments, the splicing domain is engineered to be present at, and may include, the carboxyl (C-)terminus of the targeting domain. In some embodiments, the splicing domain is located at, and may include the amino (N-)terminus of the targeting domain.

The ligand binding domain included in the targeting domain binds to or is engineered to bind to, a selected ligand. Examples include: a single chain variable fragment of an immunoglobulin, a single domain antibody, the monomeric antigen binding fragment of an antibody, the dimeric antigen binding fragment of an antibody, an scFv molecule, a synthetic Notch signal protein (SynNotch), an affibody, an alphabody, an Sso7d protein engineered to bind to a specific ligand (or similar engineered binding proteins), or any ligand-binding portion of a natural receptor molecule. In some embodiments, the ligand binding domain is located at the distal portion of the transmembrane domain, e.g., near or at the amino terminus. In some embodiments, the ligand binding domain is located at the carboxyl terminus of the transmembrane domain.

### Chimeric Receptors

Also disclosed herein are chimeric antigen receptors, such as chimeric antigen receptors, created by the protein splicing of a chimeric receptor precursor, optionally a chimeric antigen receptor precursor, and a targeting domain. In some embodiments, the splice is achieved with no inclusion of exogenous sequence from the protein splicing domains, i.e., the sequences of the protein splicing domains of both the chimeric receptor precursor and the targeting domain are completely removed in the splicing reaction. In other embodiments, the splice is achieved with minimal inclusion of exogenous sequence (scar) from the protein splicing domains. In some embodiments, the scar comprises 1, 2, 3, 4, or 5 amino acids from the protein splicing domains of the chimeric receptor precursor and/or targeting domain. Minimizing the number of amino acids in the scar can minimize an undesirable anti-chimeric receptor immune response in a patient or subject whose cells are engineered to display the chimeric receptor.

### Cells Expressing Chimeric Receptors and Precursors

Chimeric receptor precursors disclosed herein may be expressed in a cell to produce universal chimeric receptor cell precursors. In some embodiments, the cell is a human cell. In some embodiments, the human cell is produced in vitro from a human stem cell. In some embodiments, the cell is a human T-cell.

Also disclosed herein is a novel class of chimeric receptors that are synthetically created by the splicing of a chimeric receptor precursor with a targeting domain having a protein splicing domain that is compatible with and/or complementary to the protein splicing domain of the chimeric receptor precursor. The chimeric receptor precursor can be genetically engineered to be recombinantly expressed in a cell, while one or more targeting domains can be spliced onto the universal chimeric receptor precursor-expressing cell. In some embodiments, these cells are human cells. In some embodiments, the human cell is produced in vitro from a human stem cell. In some embodiments, the cells are T-cells. A single population of cells engineered to express the universal chimeric receptor precursor can be modified with diverse targeting domains. In addition, specific targeting domains can be spliced onto numerous and diverse cell populations provided that they have been engineered with an appropriate chimeric receptor precursor.

Cells that are engineered to express the chimeric receptor precursors disclosed herein can be used in a variety of ways, including treatment in which the cells are administered to a subject in need of such treatment along with one or more suitable targeting domains such that the chimeric receptor precursors in the cells engineered to express the chimeric receptor precursors splice with the targeting domains to produce in the subject cells expressing one or more chimeric receptors. Alternatively, a subject in need of treatment can be treated with specifically engineered cell therapy comprising cells modified to express a chimeric receptor precursor having customized intracellular effector function, further modified by protein splicing, to create cells with one or more specific extracellular targeting domains prior to administration to the subject.

Thus the present disclosure provides for more precisely tailored and multi-target directed cell based therapies. In alternate embodiments, the disclosure provides for more efficiently engineered single-target-directed, multi-effector-modality cell-based therapies. In addition, the present disclosure includes certain safety advantages over conventional CAR-T therapies, in that the effect of the targeting domain is limited to the cells subjected to splicing, because the constitutive genetic modification of the cells introduced to the patient, in some embodiments, will be limited to the expression of the chimeric receptor precursor only.

It is possible that the modified cells will not be able to proliferate with targeting domains, and in some embodiments it may be beneficial to prevent proliferation of the modified cells to minimize or eliminate the immune response to the precursor domain. Alternatively, once can reduce or prevent expression or the precursor domain in vivo.

In addition, the present disclosure offers advantages in terms of time to therapy, in that cells from the patient or cells from a universal cell donor can be engineered to produce the generic and universal chimeric receptor precursor, and expanded to provide a population of precursor therapeutic cells before the identity of the targeting domain has been determined. One or more targeting domains appropriate for the patient can be synthesized with the appropriate splicing domain and then the resulting targeting domain can be spliced onto the chimeric receptor precursor cells immediately prior to therapy (or as part of therapy, administered together with the cells), thus shortening the time between the determination of appropriate targeting domains and the delivery of transgenic chimeric receptor effector cell therapy to the patient. As described, the splicing may be conducted in vivo, with one or more doses of one or more targeting domain polypeptides administered to a patient after the successful establishment of a chimeric receptor precursor expressing cell population in the patient. In such embodiments, the immune response to the targeting domain polypeptides can be minimized or reduced through one or more of protein engineering of the targeting domain polypeptides, tolerization to the targeting domain polypeptides, or immunosuppressive therapy administered before, during and/or after the administration of the targeting domain polypeptides.

In some embodiments, a universal chimeric receptor cell precursor and a chimeric receptor targeting domain are contacted and spliced ex vivo. In such embodiments, an additional step can be taken to minimize or reduce immune responses by removing components of the reaction (including unspliced proteins, polypeptide fragments removed as a result of the splicing process, any splicing enzymes used, etc.) before administration of the cell therapy. In some embodiments, the cells are isolated from the reaction and cells having complete chimeric receptors are isolated by binding to immobilized ligands for the receptors. Other purification and isolation methods will be known to those skilled in the art.

Kits containing any of the disclosed chimeric receptor precursors; targeting domains; chimeric receptors; cells engineered to express the chimeric receptor precursor optionally along with one or more suitable targeting domains; cells engineered to express the chimeric receptors; and optionally additional reagents and/or instructions for use also are provided.

Also provided are nucleic acid molecules, including expression and cloning vectors, comprising the various chimeric receptor precursors, targeting domains, and chimeric receptors disclosed herein.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A chimeric receptor precursor comprising
an extracellular domain comprising a protein splicing domain,
a transmembrane domain, and
an intracellular effector domain.

2. The chimeric receptor precursor of claim 1, wherein the protein splicing domain comprises one part of a split protein-splicing domain.

3. The chimeric receptor precursor of claim 2, wherein the split protein-splicing domain is a split intein.

4. The chimeric receptor precursor of claim 3, wherein the split intein is a portion of a maxi-intein, a mini-intein, a trans-splicing intein, or an alanine intein.

5. The chimeric receptor precursor of claim 1, wherein the protein splicing domain comprises a protein or peptide that forms a spontaneous isopeptide bond.

6. The chimeric receptor precursor of claim 5, wherein the protein or peptide that forms a spontaneous isopeptide bond is SpyCatcher protein, a SpyTag peptide, a SnoopCatcher protein, a SnoopTag peptide, a SdyCatcher protein, a SdyTag peptide or a first or second portion of a split CnaB domain.

7. The chimeric receptor precursor of claim 1, wherein the protein splicing domain comprises a site recognized by a peptide ligase.

8. The chimeric receptor precursor of claim 7, wherein the peptide ligase is a sortase, a butelase, a subtiligase, a peptiligase or an omniligase.

9. The chimeric receptor precursor of any one of claims 1-8, wherein the chimeric receptor precursor is a chimeric antigen receptor precursor, an antigen receptor precursor, a cytokine receptor precursor, a growth factor receptor precursor, a chemokine receptor precursor, a hormone receptor precursor, an amino acid receptor precursor, a small molecule receptor precursor, an integrin precursor, a T-cell receptor precursor, a ligand-gated ion channel precursor, an Fc receptor precursor, or a G-protein coupled receptor precursor.

10. The chimeric receptor precursor of claim 9, wherein the chimeric receptor precursor is a chimeric antigen receptor precursor.

11. The chimeric receptor precursor of claim 10, wherein the intracellular effector domain comprises a CD3zeta domain.

12. The chimeric receptor precursor of claim 11, wherein the intracellular effector domain further comprises one or more costimulatory domains.

13. The chimeric antigen receptor precursor of claim 12, wherein the one or more costimulatory domains is selected from the group consisting of 4-1BB, CD28, CD27, ICOS, OX40, CD40, and GITR.

14. The chimeric antigen receptor precursor of claim 11, wherein the transmembrane domain is a CD28 transmembrane domain.

15. The chimeric receptor precursor of any one of claims 1-14, wherein the protein splicing domain is at the terminus of the extracellular domain distal to the transmembrane domain.

16. The chimeric receptor precursor of claim 15, wherein the protein splicing domain is at the amino terminus of the extracellular domain.

17. The chimeric receptor precursor of any one of claims 1-16, wherein the extracellular domain does not comprise a ligand binding domain.

18. The chimeric receptor precursor of any one of claims 1-16, wherein the intracellular effector domain is an intracellular signaling domain.

19. The chimeric receptor precursor of claim 18, wherein the intracellular signaling domain comprises a tyrosine kinase domain, a serine/threonine kinase domain, a histidine kinase domain, a CD3zeta domain, or a CD28 domain.

20. The chimeric receptor precursor of any one of claims 1-17, wherein the intracellular effector domain is a protein-binding domain.

21. A chimeric receptor targeting domain comprising
a ligand binding domain, and
a protein splicing domain.

22. The chimeric receptor targeting domain of claim 21, wherein the protein splicing domain comprises one part of a split protein-splicing domain.

23. The chimeric receptor targeting domain of claim 22, wherein the split protein-splicing domain is a split intein.

24. The chimeric receptor targeting domain of claim 23, wherein the split intein is a portion of a maxi-intein, a mini-intein, a trans-splicing intein, or an alanine intein.

25. The chimeric receptor targeting domain of claim 21, wherein the protein splicing domain comprises a protein or peptide that forms a spontaneous isopeptide bond.

26. The chimeric receptor targeting domain of claim 25, wherein the protein or peptide that forms a spontaneous isopeptide bond is SpyCatcher protein, a SpyTag peptide, a SnoopCatcher protein, a SnoopTag peptide, a SdyCatcher protein, a SdyTag peptide or a first or second portion of a split CnaB domain.

27. The chimeric receptor targeting domain of claim 21, wherein the protein splicing domain comprises a site recognized by a peptide ligase.

28. The chimeric receptor targeting domain of claim 27, wherein the peptide ligase is a sortase, a butelase, a subtiligase, a peptiligase or an omniligase.

29. The chimeric receptor targeting domain of any one of claims 21-28, wherein the ligand binding domain is a chimeric antigen binding domain, an antigen binding domain, a cytokine binding domain, a growth factor binding domain, a chemokine binding domain, a hormone binding domain, amino acid binding domain, small molecule binding domain, an integrin binding domain, a T-cell receptor binding domain, a ligand-gated ion channel extracellular domain, an Fc receptor binding domain, or a G-protein coupled receptor binding domain.

30. The chimeric receptor targeting domain of claim 29, wherein the antigen binding domain comprises or consists of an scFv, a single domain antibody, a Fab, a SynNotch receptor, or a chimeric protein domain.

31. The chimeric receptor targeting domain of any one of claims 21-30, wherein the protein splicing domain is at a terminus of the chimeric receptor targeting domain.

32. The chimeric receptor targeting domain claim 31, wherein the protein splicing domain is at the carboxy terminus of the chimeric receptor targeting domain.

33. The chimeric receptor targeting domain any one of claims 21-30, wherein the protein splicing domain is not at a terminus of the chimeric receptor targeting domain.

34. A chimeric receptor comprising
an extracellular domain comprising a ligand binding domain and a scar from a protein splicing reaction,
a transmembrane domain, and
an intracellular signaling domain.

35. The chimeric receptor of claim 34, wherein the protein splicing reaction is splicing of two split protein-splicing domains.

36. The chimeric receptor of claim 35, wherein the split protein-splicing domains are complementary portions of a split intein.

37. The chimeric receptor of claim 36, wherein the split inteins are complementary portions of a maxi-intein, a mini-intein, a trans-splicing intein, or an alanine intein

38. The chimeric receptor of claim 34, wherein the protein splicing reaction is spontaneous isopeptide bond formation.

39. The chimeric receptor of claim 38, wherein the spontaneous isopeptide bond formation is between a SpyCatcher protein and a SpyTag peptide, a SnoopCatcher protein and a SnoopTag peptide, a SdyCatcher protein and a SdyTag peptide, or a first and second portion of a split CnaB domain.

40. The chimeric receptor of claim 34, wherein the protein splicing reaction is splicing catalyzed by a peptide ligase.

41. The chimeric receptor of claim 40, wherein the peptide ligase is a sortase, a butelase, a subtiligase, a peptiligase or an omniligase.

42. The chimeric receptor of any one of claims 34-41, wherein the intracellular signaling domain comprises a CD3zeta domain.

43. The chimeric receptor of any one of claims 34-42, wherein the intracellular signaling domain further comprises one or more costimulatory domains.

44. The chimeric receptor of claim 43, wherein the one or more costimulatory domains is selected from the group consisting of 4-1BB, CD28, CD27, ICOS, OX40, CD40, and GITR.

45. The chimeric receptor of any one of claims 34-44, wherein the transmembrane domain is a CD28 transmembrane domain.

46. The chimeric receptor of any one of claims 34-45, wherein the antigen binding domain comprises or consists of an scFv, a single domain antibody, a Fab, a SynNotch receptor, or a chimeric protein domain.

47. The chimeric receptor of any one of claims 34-46, wherein the scar comprises 1, 2, 3, 4, or 5 amino acids.

48. The chimeric receptor of any one of claims 34-47, wherein the chimeric receptor is a chimeric antigen receptor and wherein the ligand binding domain is an antigen binding domain.

49. A universal chimeric receptor cell precursor comprising a cell engineered to express the chimeric receptor precursor of any one of claims 1-20.

50. The universal chimeric receptor cell precursor of claim 49, wherein the cell is a human cell.

51. The universal chimeric receptor cell precursor of claim 50, wherein the human cell is produced in vitro from a human stem cell.

52. The universal chimeric receptor cell precursor of any one of claims 49-51, wherein the cell is a T cell.

53. The universal chimeric receptor cell precursor of any one of claims 49-52, wherein expression of the chimeric receptor precursor is constitutive.

54. The universal chimeric receptor cell precursor of any one of claims 49-52, wherein expression of the chimeric receptor precursor is inducible.

55. A method of making the universal chimeric receptor cell precursor of any one of claims 49-52 comprising introducing into a cell a construct that expresses the chimeric receptor precursor of any one of claims 1-20.

56. The method of claim 55, wherein expression of the chimeric receptor precursor is constitutive.

57. The method of claim 55, wherein expression of the chimeric receptor precursor is inducible.

58. A cell comprising the chimeric receptor of any one of claims 34-48.

59. The cell of claim 58, wherein the cell is a human cell.

60. The cell of claim 59, wherein the human cell is produced in vitro from a human stem cell.

61. The cell of any one of claims 58-60, wherein the cell is a T cell.

62. A method of making the cell of any one of claims 58-61 comprising introducing into a cell a construct that expresses the chimeric receptor of any one of claims 34-48.

63. A method of making a cell containing a chimeric receptor comprising
contacting one or more of the universal chimeric receptor cell precursor of any one of claims 49-55 with one or more of the chimeric receptor targeting domain of any one of claims 21-32 for a time sufficient to splice or ligate together the one or more universal chimeric receptor precursor and the one or more chimeric receptor targeting domain to form a chimeric receptor cell,
wherein the one or more receptor targeting domain comprises a protein splicing domain that is complementary to the one or more protein splicing domain of the one or more universal chimeric receptor precursor, and
wherein the one or more universal chimeric receptor precursor and the one or more chimeric receptor targeting domain are spliced or ligated together via their respective protein splicing domains.

64. The method of claim 63, wherein the protein splicing domains comprise complementary portions of a split protein-splicing domain.

65. The method of claim 64, wherein the split protein-splicing domains are complementary portions of a split intein.

66. The method of claim 65, wherein the split inteins are complementary portions of a maxi-intein, a mini-intein, a trans-splicing intein, or an alanine intein.

67. The method of claim 63, wherein the protein splicing domains comprise proteins or peptides that form a spontaneous isopeptide bond.

68. The method of claim 67, wherein the proteins or peptides that form a spontaneous isopeptide bond are a SpyCatcher protein and a SpyTag peptide, a SnoopCatcher protein and a SnoopTag peptide, a SdyCatcher protein and a SdyTag peptide, or a first and second portion of a split CnaB domain.

69. The method of claim 63, wherein if the protein splicing domain comprises a site recognized by a peptide ligase and the protein splicing reaction is splicing catalyzed by a peptide ligase, further comprising contacting the universal chimeric antigen receptor T cell precursor and the chimeric antigen receptor targeting domain with a peptide ligase.

70. The method of claim 69, wherein the peptide ligase is a sortase, a butelase, a subtiligase, a peptiligase or an omniligase.

71. The method of any one of claims 63-70, wherein the cell is a human cell.

72. The method of claim 71, wherein the human cell is produced in vitro from a human stem cell.

73. The method of any one of claims 63-72, wherein the cell is a T cell.

74. The method of any one of claims F1-F7, wherein the universal chimeric receptor cell precursor and the chimeric receptor targeting domain are contacted in vitro.

75. The method of any one of claims F1-F7, wherein the universal chimeric antigen cell precursor and the chimeric receptor targeting domain are contacted in vivo.

76. The method of any one of claims F1-F7, wherein the universal chimeric receptor cell precursor and the chimeric receptor targeting domain are contacted ex vivo.

77. The method of any one of claims F1-F10, wherein more than one receptor targeting domain is spliced to one or more types of universal chimeric receptor precursor, and wherein the protein splicing domains of the more than one receptor targeting domain are orthogonal.

78. A method comprising administering to a subject in need of such treatment a cell containing a chimeric receptor of any one of claims E1-E4 or a cell containing a chimeric receptor prepared by the method of any one of claims F1-F11.

79. The method of claim G1, wherein the cell containing a chimeric receptor is a chimeric antigen receptor T (CAR-T) cell.
